(19) United States
(12) Patent Application Publication
(10) Pub. No.: US 2026/0364286 A1
(43) Pub. Date: Nov. 19, 2026

(54) APPARATUS FOR SUBSTANCE CONDITIONING AND DELIVERY TO NASAL CAVITY

(71) Applicants: James L. Orrington, II, Flossmoor, IL (US); Janie Orrington-Myers, Terre Haute, IN (US); Brandi Marie Henry, Crystal Lake, IL (US)

(72) Inventors: James L. Orrington, II, Flossmoor, IL (US); Janie Orrington-Myers, Terre Haute, IN (US); Brandi Marie Henry, Crystal Lake, IL (US)

(21) Appl. No.: 19/206,373

(22) Filed: May 13, 2025

(51) Int. Cl. A61M 15/08; A61M 16/06; A61M 16/10; A61M 21/00; A61M 21/02

(52) U.S. Cl. CPC A61M 15/085; A61M 16/0672; A61M 16/107; A61M 21/02; A61M 2021/0016; A61M 2205/7545

(57) ABSTRACT

An apparatus for substance conditioning and delivery to a nasal cavity is provided for including a substance in a gas delivered to a user and/or reducing undesirable materials from air and/or gas intended to enter a nasal cavity. The apparatus for substance conditioning and delivery to a nasal cavity may include a substance delivery apparatus, housing component, power component, fan component, filtration component, germicidal irradiation component, aroma component, nose plug component, monitoring component, cap, cannula, and delivery component.

### FIELD OF THE INVENTION

The present disclosure relates to an apparatus for substance conditioning and delivery to a nasal cavity. More particularly, the disclosure relates to purifying and/or reducing undesirable materials from gas and conditioning substances for delivery to a nasal cavity.

### BACKGROUND

For generations, and most particularly since the onset of the COVID-19 pandemic, societies have been cognizant of environmental conditions that may present health hazards. Today, vigilance in the face of air-borne viruses and other hazardous agents is prevalent throughout the world. Devices that help prevent inhalation of viruses and other harmful agents in the environment are in high demand by those seeking to maintain optimal health and not contract COVID-19 or other harmful airborne viruses and agents.

Carbon air filters are well-known to be effective in removing hazardous gaseous chemicals, as well as certain odors, from the air. HEPA (high efficiency particulate absorbing) air filters, by contrast, are mechanical in nature. HEPA filters are well-known to remove undesired particulate matter from the air. Those of skill in the art readily appreciate that carbon air filters and HEPA filters may be used together.

The sense of smell plays a significant role in the physiological effects of mood, stress, and working capacity. A large family of olfactory receptors present in humans are genetically designed to detect thousands of different fragrances. The positive impact of exposure to certain types of aromas and aromas on psychophysiological activities of humans has been recognized for years. See, e.g., K. Sowndhararajan & S. Kim, Influence of Fragrances on Human Psychophysiologylogical Activity With Special Reference to Human Electroencephalographic Response, 84(4) Sci. Pharm. 724(2016). Therefore, a need exists to solve the deficiencies present in the prior art.

What is needed is an apparatus to remove contaminants, particulates, and other undesirable elements from air prior to inhalation by a user. What is needed is an apparatus for conditioning a substance for delivery to a nasal cavity to reduce contact of undesirable elements with tissue within a nasal cavity or otherwise related to the respiratory system. What is needed is a device to remove impurities from air prior to entering the nasal cavity by performing filtration. What is needed is a portable device to be comfortably inserted into a nostril and perform air purification functions. What is needed is a substance delivery apparatus to deliver oxygen, gases, medicines, pharmaceutical formulations, aromatherapy agents, nutrients, and/or other substances to the nasal cavity of a user. What is needed is a gas delivery apparatus capable of filtering out nanoplastics that may be introduced from the delivery line.

### SUMMARY

An aspect of the disclosure advantageously provides an apparatus to remove contaminants, particulates, and other undesirable elements from air prior to inhalation by a user. An aspect of the disclosure advantageously provides an apparatus for conditioning a substance for delivery to a nasal cavity to reduce contact of undesirable elements with tissue within a nasal cavity or otherwise related to the respiratory system. An aspect of the disclosure advantageously provides a device to remove impurities from air prior to entering the nasal cavity by performing filtration. An aspect of the disclosure advantageously provides a portable device to be comfortably inserted into a nostril and perform air purification functions. An aspect of the disclosure advantageously provides a substance delivery apparatus to deliver oxygen, gases, medicines, pharmaceutical formulations, aromatherapy agents, nutrients, and/or other substances to the nasal cavity of a user. An aspect of the disclosure advantageously provides a gas delivery apparatus capable of filtering out nanoplastics that may be introduced from the delivery line.

Accordingly, an embodiment of the invention may be provided by a substance delivery apparatus for nasal use comprising a housing, nose plug component, and delivery component. The housing may substantially enclose an interior space. The nose plug component may be operatively attached to a nose end of the housing to be removably inserted into and form an at least partial seal with a nostril such that a substance comprising a medicinal product is directed to a nasal cavity through the nose plug component via the nostril. The delivery component may be located within the interior space to condition the substance and deliver the substance that is conditioned to the nasal cavity via the nose plug component.

In another aspect, conditioning the substance may include mixing the substance with a gas. The apparatus may further include a cap, a gas treatment component, and a cannula of the cap. The cap may be removably attached to a cap end of the housing to receive the gas delivered from a gas source via a delivery line, the cap end being distal to the nose end. The gas treatment component may be located within the interior space to at least partially purify the gas for delivery to the nasal cavity comprising a filtration component to remove impurities from the gas. The cannula of the cap may be configured to receive the delivery line. The delivery line may have a delivery line diameter configured to be selectively installed over the cannula such that the gas from the delivery line is received through a gas receiving aperture of the cannula.

In another aspect, the filtration component may condition the gas to at least partially filter nanoplastics from the gas received via the delivery line. The nanoplastics being filtered by the filtration component may have at least one dimension measuring less than 100 nanometers.

In another aspect, the gas may be pressurized and substantially comprised of oxygen.

In another aspect, the oxygen may originate from an on-board oxygen generation system of an aeronautical vehicle.

In another aspect, the gas may be pressurized and may originate from a continuous positive airway pressure (CPAP) machine.

In another aspect, the substance may include a physical-chemical identifier. The medicinal product may be stored via a solid dosage form. At least part of the medicinal product may be extracted from the solid dosage form to be mixed with a gas in the delivery component to be delivered to the nasal cavity via the nose plug.

In another aspect, the medicinal product may be at least partially incorporated into the substance via a multi-step process of pharmaceutical formulation including the steps: mixing active drug ingredients, mixing an excipient with the active ingredients to create the medicinal product, and mixing the medical product with the gas to condition the substance for delivery to the nasal cavity.

In another aspect, the pharmaceutical formulation may further include the steps: mixing a pH adjuster composition with the medicinal product to condition a pH level of the substance, and creating the solid dosage form of the medicinal product from which the substance is delivered with the gas through the nose plug component that has been inserted into to the nostril.

In another aspect, the pharmaceutical formulation may further include the step of storing the active drug ingredients and the excipient that are mixed as the medicinal product in the solid dosage form.

In another aspect, the pharmaceutical formulation may further include the step of incorporating the substance in nanofibers via electrostatic spinning.

In another aspect, the pharmaceutical formulation may further include the step of storing the active drug ingredients and the excipient that are mixed with a physical-chemical identifier as the medicinal product in the solid dosage form. The physical-chemical identifier may initiate a psychological response in a patient after being received by the nasal cavity via the nose plug component.

In another aspect, the physical-chemical identifier may include an aromatherapy agent.

In another aspect, the physical-chemical identifier may include a molecular taggant.

In another aspect, the substance may include nutrients as absorption enhancers. An efficacy of delivery of the substance via the nasal cavity may be improved via the nutrients.

Accordingly, an embodiment of the invention may be provided for a substance delivery apparatus for nasal use that may include an interior space, a nose plug component, a delivery component, a cap, a gas treatment component, and a cannula. The interior space may be substantially enclosed by a housing. The nose plug component may be operatively attached to a nose end of the housing to be removably inserted into and form an at least partial seal with a nostril such that a substance comprising a medicinal product is directed to a nasal cavity through the nose plug component via the nostril. The delivery component may be located within the interior space to condition the substance and deliver the substance that is conditioned to the nasal cavity via the nose plug component. The cap may be removably attached to a cap end of the housing to receive the gas delivered from a gas source via a delivery line, the cap end being distal to the nose end. The gas treatment component may be located within the interior space to at least partially purify the gas for delivery to the nasal cavity comprising a filtration component to remove impurities from the gas. The cannula of the cap may receive the delivery line. The substance may further include a physical-chemical identifier. The medicinal product may be stored via a solid dosage form. At least part of the medicinal product may be extracted from the solid dosage form to be mixed with a gas in the delivery component to be delivered to the nasal cavity via the nose plug.

In another aspect, conditioning the substance may include mixing the substance with a gas. The filtration component may condition the gas to at least partially filter nanoplastics from the gas received via the delivery line. The nanoplastics being filtered by the filtration component may have at least one dimension measuring less than 100 nanometers.

In another aspect, the medicinal product may be at least partially incorporated into the substance via a multi-step process of pharmaceutical formulation comprising the steps mixing active drug ingredients, mixing an excipient with the active ingredients to create the medicinal product, mixing the medical product with the gas to condition the substance for delivery to the nasal cavity, and storing the active drug ingredients and the excipient that are mixed as the medicinal product in the solid dosage form.

In another aspect, the pharmaceutical formulation may further include the step of incorporating the substance into nanofibers via electrostatic spinning.

In another aspect, the substance may include nutrients as absorption enhancers. An efficacy of delivery of the substance via the nasal cavity may be improved via the nutrients.

Terms and expressions used throughout this disclosure are to be interpreted broadly. Terms are intended to be understood respective to the definitions provided by this specification. Technical dictionaries and common meanings understood within the applicable art are intended to supplement these definitions. In instances where no suitable definition can be determined from the specification or technical dictionaries, such terms should be understood according to their plain and common meaning. However, any definitions provided by the specification will govern above all other sources.

Various objects, features, aspects, and advantages described by this disclosure will become more apparent from the following detailed description, along with the accompanying drawings in which like numerals represent like components.

### BRIEF DESCRIPTION OF THE DRAWINGS

FIG. 1 is a block diagram view of an illustrative apparatus for substance conditioning and delivery to a nasal cavity, according to an embodiment of this disclosure.

FIG. 2 is a side elevation view of an illustrative apparatus for substance conditioning and delivery to a nasal cavity, according to an embodiment of this disclosure.

FIG. 3 is a top plan view of an illustrative apparatus for substance conditioning and delivery to a nasal cavity, according to an embodiment of this disclosure.

FIG. 4 is a cross-sectional view of an illustrative apparatus for substance conditioning and delivery to a nasal cavity shown in FIG. 3, according to an embodiment of this disclosure.

FIG. 5 is a perspective view of an illustrative apparatus for substance conditioning and delivery to a nasal cavity, delivery line, and gas source, according to an embodiment of this disclosure.

FIG. 6 is a perspective view of an illustrative use of a substance delivery and filtration apparatus enabled by this disclosure.

FIG. 7 is a perspective view of an example solid dosage form that may be used with an illustrative apparatus for substance conditioning and delivery to a nasal cavity, according to an embodiment of this disclosure.

FIG. 8 is a perspective view of an alternative example solid dosage form with a pH adjustment composition that may be used with an illustrative apparatus for substance conditioning and delivery to a nasal cavity, according to an embodiment of this disclosure.

FIG. 9 is a perspective view of an alternative example solid dosage form with nutrients that may be used with an illustrative apparatus for substance conditioning and delivery to a nasal cavity, according to an embodiment of this disclosure.

FIG. 10 is a perspective view of an alternative example solid dosage form with a molecular taggant that may be used with an illustrative apparatus for substance conditioning and delivery to a nasal cavity, according to an embodiment of this disclosure.

FIG. 11 is a flowchart view of an illustrative operation for conditioning a substance for delivery to a nasal cavity, according to an embodiment of this disclosure.

### DETAILED DESCRIPTION

The following disclosure is provided to describe various embodiments of an apparatus for substance conditioning and delivery to a nasal cavity. Skilled artisans will appreciate additional embodiments and uses of the present invention that extend beyond the examples of this disclosure. Terms included by any claim are to be interpreted as defined within this disclosure. Singular forms should be read to contemplate and disclose plural alternatives. Similarly, plural forms should be read to contemplate and disclose singular alternatives. Conjunctions should be read as inclusive except where stated otherwise.

Expressions such as “at least one of A, B, and C” should be read to permit any of A, B, or C singularly or in combination with the remaining elements. Additionally, such groups may include multiple instances of one or more element in that group, which may be included with other elements of the group. All numbers, measurements, and values are given as approximations unless expressly stated otherwise.

For the purpose of clearly describing the components and features discussed throughout this disclosure, some frequently used terms will now be defined, without limitation. The term “HEPA filter,” as it is used throughout this disclosure, is defined as a high-efficiency particulate absorbing filter being, using, or containing a filter usually designed to capture a substantial portion of airborne particles, for example 99.97 percent of airborne particles measuring 0.3 micrometers or greater in diameter, from air passing through the filter. The term “nasal,” as it is used throughout this disclosure, is defined as pertaining to the nose and related features. The term “nostril,” as it is used throughout this disclosure, is defined as an opening to a nasal cavity. The term “medicinal,” as it is used throughout this disclosure, is defined as exhibiting therapeutic properties, including properties useful for curing disease, treating an illness or condition, and/or relieving pain.

The term “cannula,” as it is used throughout this disclosure, is defined as a small variably sized tube that may assist in guiding air and/or gases. The term “microorganisms,” as it is used throughout this disclosure, is defined as an organism of microscopic size, including, but not limited to, bacterium, virus, or fungi. The term “gas,” as it is used throughout this disclosure, is defined as a substance that has neither independent shape nor volume and holds the property of indefinite expansion, including, but not limited to, molecules (such as oxygen, radon, and nitrogen), chemical compounds (such as nitrogen dioxide, methanal, and benzene), and mixtures of various molecules and chemical compounds (such as air, biogas, and natural gas). The term “continuous positive airway pressure (CPAP) machine,” as it is used throughout this disclosure is defined as a gas source that can be used to deliver a continuous flow of pressurized gas to the nasal cavity. The term “aeronautical vehicle,” as it is used throughout this disclosure is defined as a vehicle capable of traveling above ground level, for example and without limitation, an airplane, military air vehicle, helicopter, spaceship, or other such vehicles.

The term “nonbiodegradable polymer,” as it is used throughout this disclosure is defined as a type of plastic or other material that cannot be broken down by natural processes or microbes. The term “nanoplastics,” as it is used throughout this disclosure is defined as tiny plastic particles with dimensions ranging from about 1 to 100 nanometers, which may pose potential environmental and health risks due to their small size and ability to penetrate cells. The term “nanofibers,” as it is used throughout this disclosure is defined as extremely thin fibers with diameters in the nanometer range, often used in applications like filtration.

The term “pharmaceutical formulation,” as it is used throughout this disclosure is defined as a formulation of a medicine or medicinal product, which may be designed to deliver the active drug to the body in a safe and effective way, which can include tablets, capsules, injections, or creams, without limitation. The term “nutrients,” as it is used throughout this disclosure is defined as substances that provide nourishment essential for growth, maintenance, and repair of the body, including proteins, carbohydrates, fats, vitamins, and minerals.

Various aspects of the present disclosure will now be described in detail, without limitation. In the following disclosure, an apparatus for substance conditioning and delivery to a nasal cavity will be discussed. Those of skill in the art will appreciate alternative labeling of the apparatus for substance conditioning and delivery to a nasal cavity as a nose filter device, the substance conditioning and delivery apparatus, nasal filtration device, air purification apparatus for nasal application, portable powered air purification apparatus, the invention, the apparatus, or other similar names. Similarly, those of skill in the art will appreciate alternative labeling of the apparatus for substance conditioning and delivery to a nasal cavity as an air filtration operation, substance delivery and/or filtration technique, gas quality improvement method, method, operation, the invention, or other similar names. Skilled readers should not view the inclusion of any alternative labels as limiting in any way.

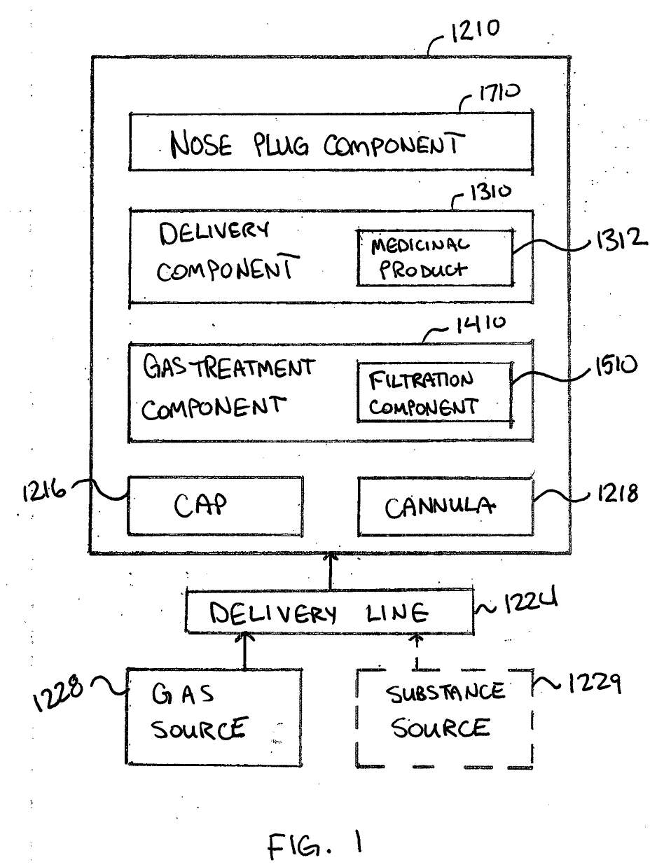

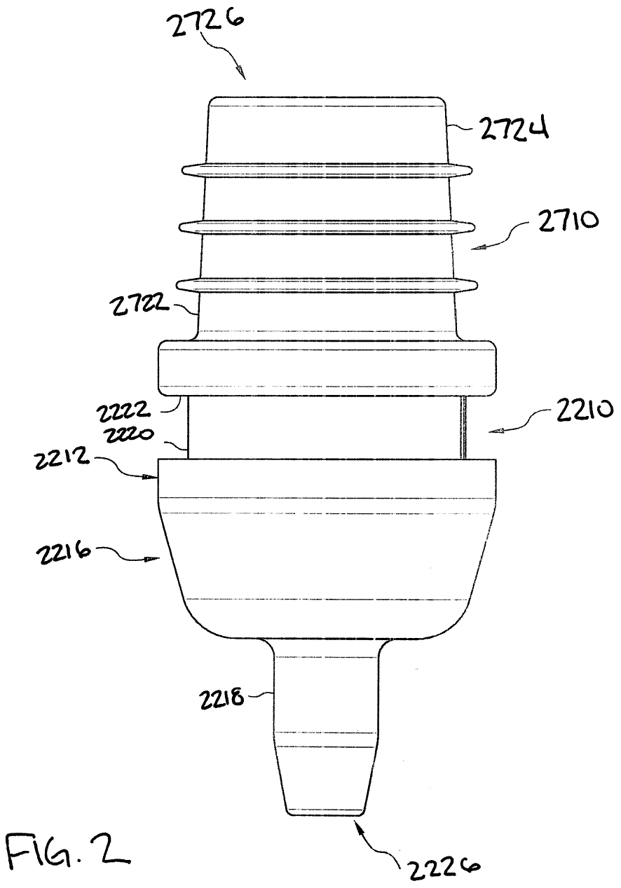

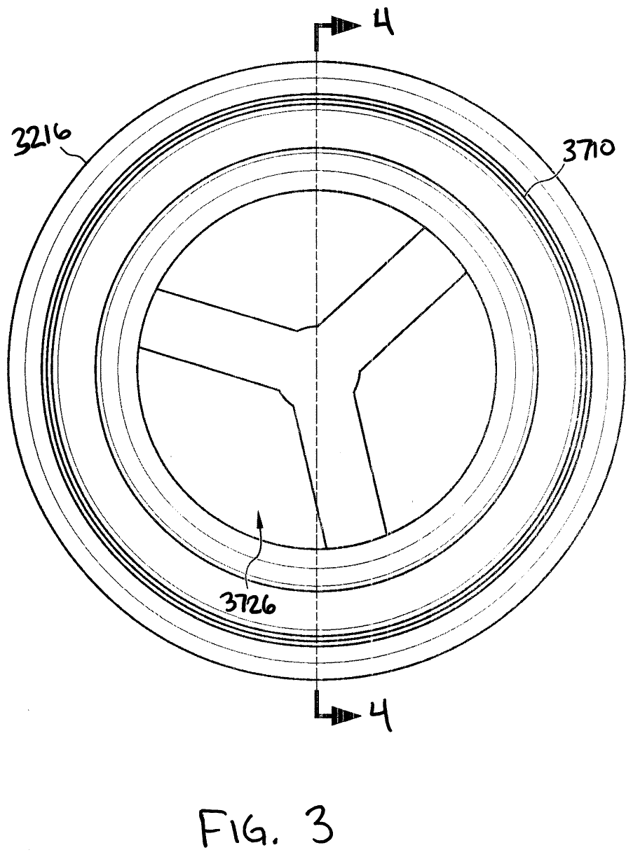

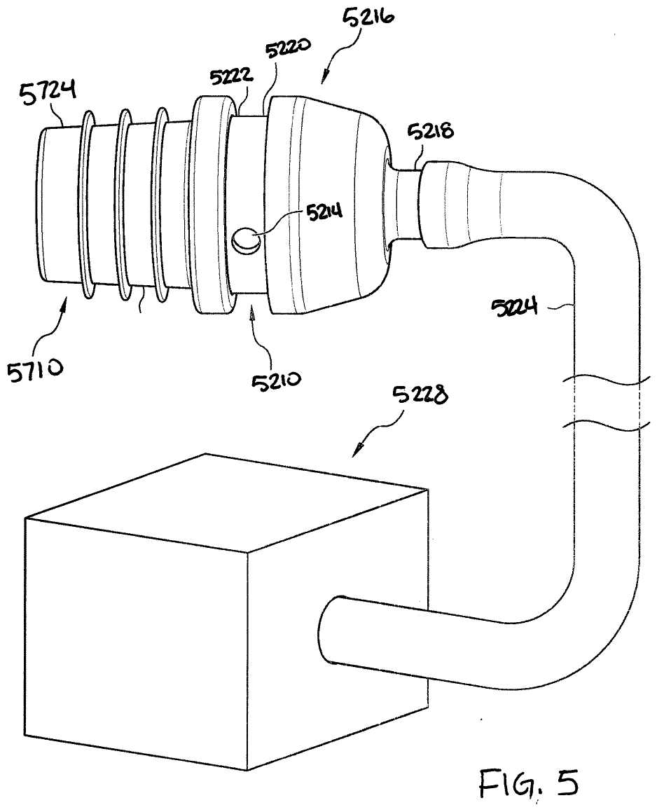

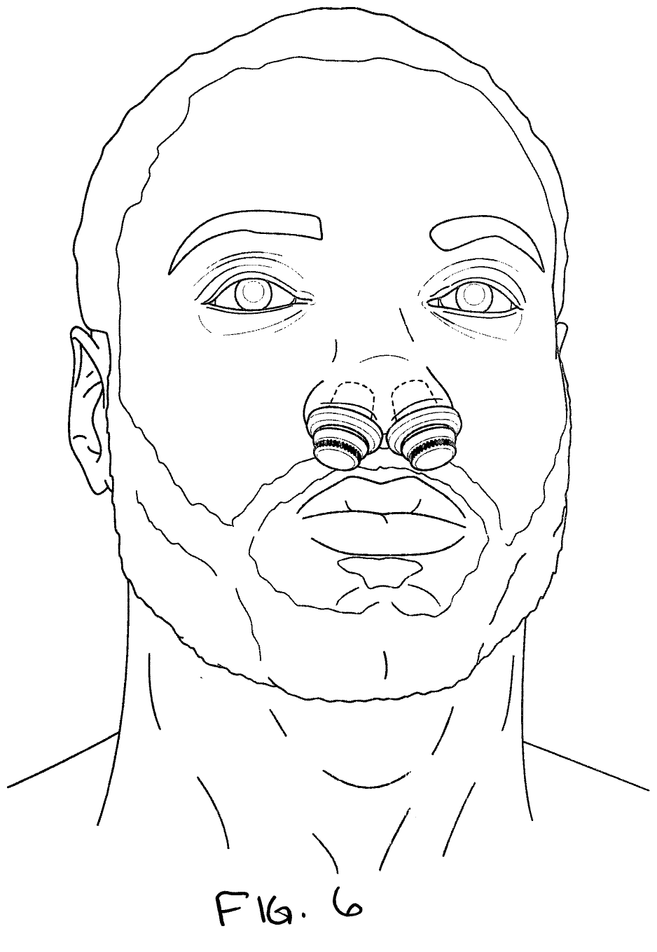

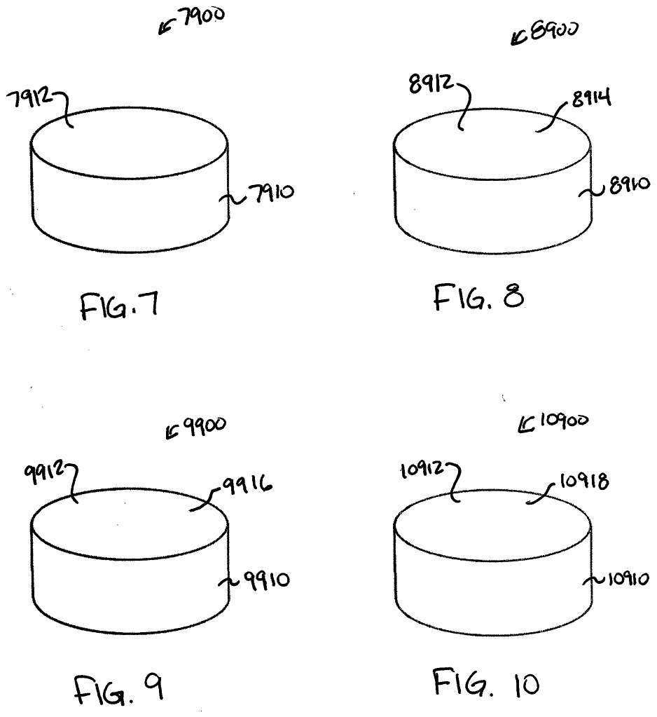

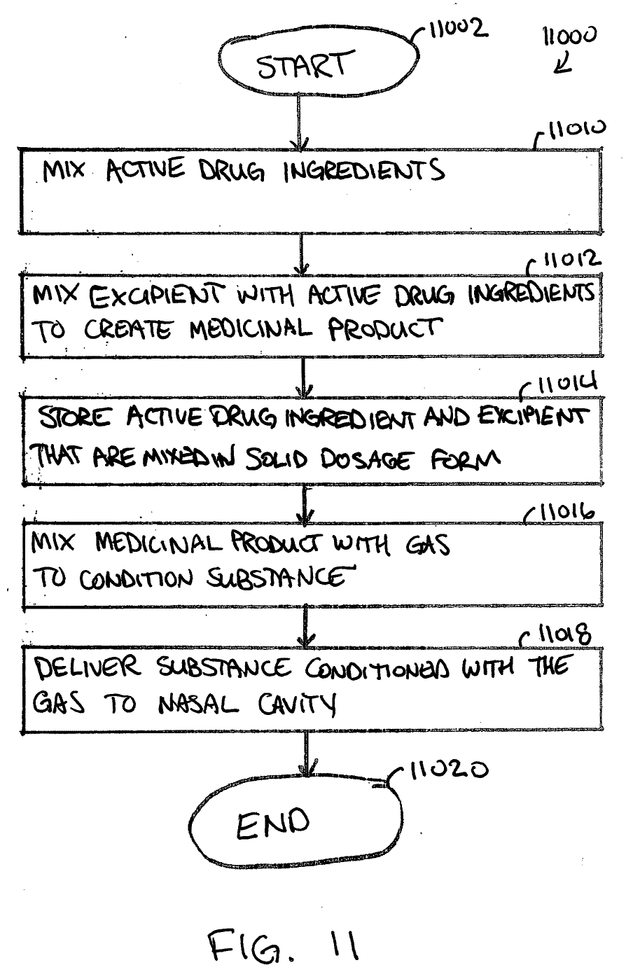

Referring now to FIGS. 1-11, an apparatus for conditioning a substance for delivery to a nasal cavity will now be discussed in more detail. Persons skilled in the art will appreciate disclosure that enables the use of the apparatus for passage of air may additionally disclose and enable uses of the apparatus for passage of gas. The use of air in any of the following discussion, examples, and other enabling context is intended to additionally enable use for other gases in replacement of or addition to air, without limitation. The apparatus for conditioning a substance for delivery to a nasal cavity may include a housing, nose plug component, delivery component, medicinal product, gas treatment component, filtration component, cap, cannula, and additional components that will be discussed in greater detail below. The apparatus for conditioning a substance for delivery to a nasal cavity may operate one or more of these components interactively with other components for purifying and/or reducing undesirable materials from air and/or other gasses intended to enter a nasal cavity.

In the interest of clearly presenting the invention, the following disclosure will be collectively discussed in the context of various illustrative embodiments. Skilled artisans should appreciate that features discussed in the context of one embodiment may be included in additional embodiments, even if not expressly stated when discussing such embodiments, and thus should view the teachings of this disclosure as a whole and not as a collection of disparate embodiments or otherwise limiting.

The apparatus for conditioning a substance for delivery to a nasal cavity will now be discussed. FIGS. 1-11 highlight examples of the apparatus for conditioning a substance for delivery to a nasal cavity in various embodiments. The apparatus for conditioning a substance for delivery to a nasal cavity may include various components that collectively provide an improved capability to advantageously remove pathogens, contaminants, particulates, and/or other undesirable elements from a gas and/or condition a substance for delivery to a user before the gas may enter the respiratory system of a user. In some embodiments, the apparatus for conditioning a substance for delivery to a nasal cavity may include various components located within a housing 1210, 2210, 4210, 5210. Such components may include, without limitation, gas treatment components 1410, 4410, a nose plug component 1710, 2710, 3710,710, 5710, and other components. Gas treatment components 1410, 4410 may include various aspects to improve the quality of air before it is received by a user, such aspects including without limitation, a filtration component, a gas treatment component, a delivery component, and/or other components that will be apparent to a person of skill in the art after having the benefit of this disclosure. Each of these components will be discussed in greater detail throughout this disclosure in the context of various embodiments. Skilled artisans should appreciate that each aspect may be included by alternative embodiments.

The housing will now be discussed in greater detail. FIGS. 1-2 and 4-6 highlight examples of the housing, which may also be shown in other figures. The housing 1210, 2210, 4210, 5210 may be provided to substantially enclose an interior space within an apparatus for conditioning a substance for delivery to a nasal cavity. The housing 1210, 2210, 4210, 5210 may provide an enclosure to at least partially surround some of the functional components of an apparatus for conditioning a substance for delivery to a nasal cavity within an interior space defined within the housing.

In an illustrative structural configuration, the housing 1210, 2210, 4210, 5210 may be at least partially comprised of plastic, without limitation. The housing may have virtually any diameter and height to preserve operability of gas treatment components. In one embodiment, the housing may have a substantially cylindrical shape, for example, approximately 10 millimeters and approximately 30 millimeters in diameter, without limitation. The housing may have a height sufficient to enclose the gas treatment components, for example, between approximately 7 millimeters and approximately 14 millimeters, without limitation.

The cylindrical shape may advantageously correspond with a nostril of a user for improved comfort, fit, and seal upon insertion into the nostril. Additionally, the use of a cylindrical shape may advantageously assist with the removal of an apparatus for conditioning a substance for delivery to a nasal cavity enabled by this disclosure when use of such apparatus is no longer desired. In additional embodiments, the housing may be provided in a non-cylindrical shape, without limitation. For example, at least part of the housing may be provided by a rectangular shape, conical shape, rounded shape, and/or another shape that would be appreciated by those of skill in the art after having the benefit of this disclosure. In the example of a rectangular shape, the housing may be between approximately 10 millimeters and approximately 30 millimeters in width and between approximately 7 millimeters and approximately 14 millimeters in height, without limitation.

The housing 1210, 2210, 4210, 5210 may be constructed using various parts, for example a housing cap end 2220, 4220, 5220, a housing nose end 2222, 4222, 5222, and/or other portions that will be apparent after having the benefit of this disclosure. The housing 1210, 2210, 4210, 5210 may be sized with sufficient interior space to receive, hold, and allow operation of additional components including delivery components 1310, 4310; gas treatment components 1410, 4410; and/or other components that may be included within the interior space.

In the same or other embodiments, the housing 1210, 2210, 4210, 5210 may additionally comprise a cap end 2220, 4220, 5220 of the housing and a nose end 2222, 4222, 5222 of the housing. The cap end 2220, 4220, 5220 may be on the opposite side of the nose end 2222, 4222, 5222 of the housing. The housing 1210, 2210, 4210, 5210 may include access points to allow interaction with the components included within the interior space of the housing from an exterior of the housing.

In various embodiments, the housing may further comprise an auxiliary aperture 5214 on the housing 1210, 2210, 4210, 5210. The auxiliary aperture 5214 may be located on the wall of the housing. Additionally, the auxiliary aperture 5214 may be of virtually any size. Those skilled in the art may realize the size and location of the auxiliary aperture after having the benefit of this disclosure. Furthermore, the auxiliary aperture 5214 may hold various purposes for the apparatus, including, but not limited to, pressure relief, moisture relief, an access point, and/or other purposes that a person skilled in the art would realize after having the benefit of this disclosure. Some of these purposes will be discussed in greater detail throughout this disclosure.

As previously mentioned, the auxiliary aperture 5214 may be used for moisture relief. In various embodiments, the housing 1210, 2210, 4210, 5210 may additionally include a drying agent. The drying agent may at least partially remove moisture within the interior space of the housing 1210, 2210, 4210, 5210. In the same or different embodiments, the moisture collected by the drying agent may be at least partially vented out through the auxiliary aperture 5214. Drying agents include, but are not limited to, calcium chloride, calcium oxide, sodium sulfate, sulphuric acid, magnesium sulfate, and/or other drying agents. Those skilled in the art will appreciate the various types of drying agents, whether chemical or mechanical, that may be used in the apparatus, after having the benefit of this disclosure.

The housing 1210, 2210, 4210, 5210 may additionally include an interface to receive and/or hold a nose plug component 1710, 2710, 3710, 4710, 5710, which may be distal to the bottom surface of the housing. The nose plug component 1710, 2710, 3710, 4710, 5710 will be discussed in greater detail later in this disclosure. In this embodiment, the housing 1210, 2210, 4210, 5210 may include features to apply friction to the nose plug component 1710, 2710, 3710, 4710, 5710, textured aspects or protrusions to receive corresponding depressions on an interior surface of the nose plug component 1710, 2710, 3710, 4710, 5710, or otherwise be configured to operatively hold the nose plug component 1710, 2710, 3710, 4710, 5710 to the housing 1210, 2210, 4210, 5210 in a removably installed configuration. Additionally, in the same or other embodiments, the nose plug component 1710, 2710, 3710, 4710, 5710 may include textured aspects, protrusions, or ridges on the exterior which may assist in applying friction to at least part of the nostril when the apparatus is in use. In an exemplary embodiment, the nose plug component 1210, 2210, 3210, 4210 may include three circumferential ridges, without limitation. However, those skilled in the art may appreciate the various number of ridges that may be included on the nose plug component, after having the benefit of this disclosure.

In the same or other embodiments, the apparatus for conditioning a substance for delivery to a nasal cavity may additionally include a cap 1216, 2216, 3216, 4216, 5216. The cap 1216, 2216, 3216, 4216, 5216 may advantageously provide a male fitting for a delivery line to a substance source 1229 and/or gas source 1228, 5228, for example, via a cannula 1218, 2218, 4218, 5218. The delivery line and substance source and/or gas source will be discussed in greater detail below.

The cap 1216, 2216, 3216, 4216, 5216 may be removably attached to the housing 1210, 2210, 4210, 5210 via a cap receiving feature 2212, 4212 of the housing 1210, 2210, 4210, 5210, which will be discussed in greater detail below. Generally, the cap 1216, 2216, 3216, 4216, 5216 may assist in direct administration of substances, for example a gas and/or medicinal product 1312, via a substance or gas receiving aperture 2226, 4226, into at least part of the housing 1210, 2210, 4210, 5210. In an illustrative structural configuration, the aperture may be located virtually anywhere on the cap. In some embodiments, the aperture may be located on the opposite side of the cap receiving feature 2212, 4212. In the same or other embodiments, the substance or gas receiving aperture 2226, 4226 may be located on the cannula 1218, 2218, 4218, 5218, which will be discussed in greater detail below. Additionally, the substance or gas receiving aperture may be of virtually any shape and of any size, as will be appreciated by those of skill in the art, after having the benefit of this disclosure.

In one or more embodiments, the cap 1216, 2216, 3216, 4216, 5216 may additionally comprise a cannula 1218, 2218, 4218, 5218. In some embodiments, the cannula may be located on one end of the cap 1216, 2216, 3216, 4216, 5216. The cannula 1218, 2218, 4218, 5218 may have a cannula diameter. The cannula 1218, 2218, 4218, 5218 may have a cannula male end which may advantageously be used to fit into the end of a delivery line 1224, 5224. The delivery line 1224, 5224 may have a delivery line diameter. The delivery line 1224, 5224 may be used to connect the cannula 1218, 2218, 4218, 5218 to a substance source 1229 and/or gas source 1228, 5228. In embodiments, the cannula diameter may be smaller than the delivery line diameter.

In embodiments, the apparatus may additionally include a gas source 1228, 5228 from which gas may be released or provided. Examples of a gas source 1228, 5228 may include a pressurized tank or a continuous positive airway pressure machine, an on-board oxygen generator system of an aeronautical vehicle, pure oxygen delivery mechanism, air delivery system from fighter jets or other military vehicles, oxygen and/or air delivery system from spacecraft or other vehicles intended to approach or pass the upper atmosphere, a pressurized tank, or other gas sources, without limitation.

In additional embodiments, the apparatus may additionally include a substance source 1229 from which a substance may be released from or provided. Examples of a substance source 1229 may include a pressurized tank, an aeronautical vehicle air supply system, medical device, or accessory for a continuous positive airway pressure machine, without limitation. Furthermore, examples of substance source 1229 may include a reservoir 5230 and or space that may include a solid dose form 7900, 8900, 9900, 10900 of the substance 7912, 8912, 9912, 10912 held by nanofibers 5231, or other form of substance delivery, without limitation.

Additionally, the gas source 1228, 5228 and/or substance source 1229 may be attached to a delivery line 1224, 5224. The delivery line 1224, 5224 may advantageously assist in delivering a gas and/or substance from the substance source 1229 and/or gas source 1228, 5228 to the apparatus. For example, a substance 5241 may be held by the substance source 1229, from which it may be delivered to the apparatus via the delivery line 1224, 5224. As previously mentioned, the delivery line 1224, 5224 may have a delivery line diameter. The delivery line 1224, 5224 may be selectively installed to the cap, so that the gas and/or substance from the delivery line may be received through the gas and/or substance receiving aperture 2226, 5226 of the cap 1216, 2216, 3216, 4216, 5216. In various embodiments, the delivery line 1224, 5224 may be made of plastic or rubber, without limitation. Furthermore, the air inside the delivery line 1224, 5224 may enter the cannula 1218, 2218, 4218, 5218 via the gas and/or substance receiving aperture 5226. As the gas continues from the cannula 1218, 2218, 4218, 5218 into the cap 1216, 2216, 3216, 4216, 5216, the effective diameter of the cap may increase. This increase of the diameter may slow the velocity of the gas provided by the gas source and/or substance provided by the substance source. The increase of the diameter may also increase the efficacy of filtration when the air is delivered through the filtration component 4510. Those people skilled in the art will appreciate the various benefits that one may find from an increase in the diameter from the delivery line to the cannula and to the cap.

In embodiments, the housing 1210, 2210, 4210, 5210 may comprise a cap receiving feature 2212, 4212 which may advantageously assist in temporarily installing the cap 1216, 2216, 3216, 4216, 5216 to the housing 1210, 2210, 4210, 5210. In the various embodiments, the cap receiving feature 2212, 4212 may be at least partially flexible to allow for installation and/or removal of the cap 1216, 2216, 3216, 4216, 5216. The cap receiving feature 2212, 4212 may be provided near the cap end 2220, 4220, 5220 of the housing 1210, 2210, 4210, 5210. In various embodiments, the cap receiving feature 2212, 4212 includes a circumferential ridge around the cap end 2220, 4220, 5220 of the housing 1210, 2210, 4210, 5210. In other embodiments, the cap receiving feature 2212, 4212 includes a partial rim on one or more sides of the cap end 2220, 4220, 5220 of the housing 1210, 2210, 4210, 5210. Those of skill in the art will appreciate the various mechanical structures that may be included in the cap receiving feature 2212, 4212 to allow for the temporary installation and/or removal of the cap 1216, 2216, 3216, 4216, 5216 on the housing 1210, 2210, 4210, 5210 or any other part of the apparatus.

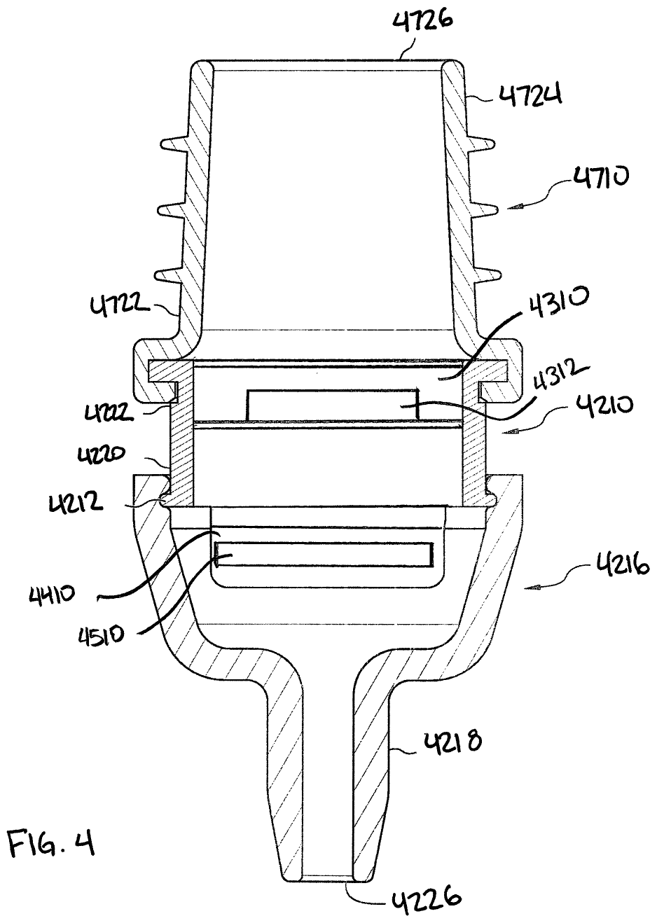

The gas treatment component will now be discussed in greater detail. FIGS. 1 and 4 highlight examples of the gas treatment component, which may also be shown in other figures. Various components included by the gas treatment component 1410, 4410 will be discussed in greater detail throughout this disclosure. Gas treatment component 1410, 4410 may interact with air and other gases flowing through an interior space of an apparatus for conditioning a substance for delivery to a nasal cavity, such as one enabled by this disclosure to change a characteristic of such air or other gas. In various embodiments, the gas treatment component 1410, 4410 may include a filtration component 1510, 4510 to remove particulates, pathogens, microorganisms, and other undesirable elements from the air as it passes through the interior space. The gas treatment component may additionally condition a gas to include a substance such as a medical product, which may be delivered to a nasal cavity of a user. The substances may include medicinal products, physical-chemical identifiers such as aromas, pH adjustment compositions, nutrients, molecular taggants, and other contents that would be apparent to a person of skill in the art after having the benefit of this disclosure. Examples of some of the embodiments will be provided in greater detail below.

The gas treatment component may include a filtration component 1510, 4510, which may include various filters and other elements to affect the composition of gas and air passing through or around the filtration component. Generally, air or other gases passed through the filtration component 1510, 4510 may be directed to the nose plug component. In one embodiment, the filtration component 1510, 4510 may include a carbon filter to advantageously remove undesired particulates, impurities, including microorganisms, and other materials from air passing through such carbon filter. In one example, provided without limitation, a carbon filter may be provided with a thickness of between approximately 5 millimeters and approximately 10 millimeters. In some embodiments, the carbon filter may include activated carbon, as will be appreciated by those of skill in the art. In some embodiments, the filtration component 1510, 4510 may at least partially filter nanoplastics and other nanoparticles from the gas, for example, which may have been received from the delivery line during transmission of the gas or other substance.

The carbon filter may reduce the presence of airborne pathogens, including viruses and bacteria, microplastics, and other impurities from entering the respiratory system. The carbon filter may filter gaseous volatile organic compounds and other undesirable substances, such as cigarette smoke, drying paint, benzene, toluene, xylene, nanoplastics, and/or other substances, without limitation. Carbon filtration may additionally filter unpleasant odors from air and may contribute to decreased stress and increased calmness and feelings of well-being on the part of such user. The carbon filter may include activated carbon.

In another embodiment, the filtration device may include a high efficiency particulate absorbing (HEPA) filter, as will be appreciated by those of skill in the art. In one example, provided without limitation, a HEPA filter may be provided having a wafer configuration with a thickness of between approximately 5 millimeters and approximately 10 millimeters. The HEPA filter may reduce potentially harmful agents and contaminants such as air bacteria, dust, pollen, mold, nanoplastics, and other types of airborne particles. As will be appreciated by those of skill in the art, HEPA filtration may remove at least approximately 98% of airborne contaminants having a size of approximately 0.3 microns or more. Additionally, the HEPA filter may be replaceable by the user.

In some embodiments, either the carbon filter or the HEPA filter may be provided as the sole filtering technique included by the filtering component. In other embodiments, a two-stage filtering process may be provided whereby air is moved through the carbon filter for a first filtering pass and then through the HEPA filter for an additional filtering pass. In some embodiments, the filter may be scented or otherwise include and/or provide a substance capable of interacting with an olfactory nerve, such as a physical-chemical identifier, without limitation. Those of skill in the art will appreciate additional filter types that may be included, which may also be arranged by the filtering component to affect the removal of particulates, pathogens, and other undesirable elements from the air passing through the filtration component 1510, 4510.

The delivery component will now be discussed in greater detail. FIGS. 1, 4, and 7-10 highlight an example of the delivery component, which may also be shown in other figures. Inclusion of a delivery component 1310, 4110 may advantageously enhance a user's health, thus decreasing a user's susceptibility to reacting negatively to various symptoms. The delivery component may, without limitation, be located within a housing 1210, 2210, 4210, 5210 as contemplated herein. In an embodiment, the delivery component may be located subsequent to the filtration component 4510, such as to receive the gas after it has passed through the filtration component 1510, 4510 and other aspects of the gas treatment component 1410, 4110. In other embodiments, the delivery component may be located before the gas treatment component 1410, 4410, in the housing 1210, 2210, 4210, 5210.

In one embodiment, a delivery component 1310, 4110 may be included such as to introduce a medicinal product 1312, 4312 into gas passing through the interior space. The delivery component 1310, 4110 may advantageously apply various medicinal products, aromas, and other substances in different amounts and/or in different forms, such as a solid dose form 7900, 8900, 9900, 10900, aerosol, or other form, without limitation. Such substances 7912, 8912, 9912, 10912 may include drugs suitable for intranasal administration, which may include, without limitation, antiepileptics, sedatives, opiate analgesics and antagonists, local anesthetics, epistaxis control agents, and anti-hypoglycemic agents.

The delivery component 1310, 4110 may include a space to hold a solid dosage form, which may include the medical product and/or additional contents for conditioning a gas for delivery to a nasal cavity. Substances including medicinal products and/or other contents may be at least partially held by nanofibers as a solid dosage form. Nonbiodegradable polymers may advantageously be used to create nanofibers via electrospinning, and may include materials chosen when long-term stability, durability, or specific physical/chemical properties are required, and degradation is undesirable for the application. In some embodiments, nanofibers may be made of a nonbiodegradable polymer that may include, without limitation, polyacrylonitrile, polyurethane, polymethyl methacrylate, polystyrene, polyvinyl chloride, polyethylene terephthalate, nylon, polyvinylidene fluoride and its copolymers, polyethersulfone, polysulfone, and other nonbiodegradable polymers that would be apparent to a person of skill in the art after having the benefit of this disclosure.

In one embodiment, the substances may be incorporated into the nanofibers via electrostatic spinning. For example, a substance such as an active drug ingredient, growth factor, catalyst, or other functional material may be incorporated into nanofibers using electrostatic spinning, also referred to as electrospinning without limitation. For example, a substance may be dissolved or stably suspended as fine nanoparticles into the polymer solution before electrospinning by blending. The polymers and the substance may be mixed thoroughly in a suitable solvent or solvent mixture to create a homogeneous fluid. This homogeneous mixture, solution, or suspension may then be electrospun. A high voltage may be applied as the fluid is pumped through a spinneret, and the solvent may evaporate during the fiber formation process.

In another example of electrostatic spinning, emulsion may be used when the substance is immiscible with the primary polymer solution. An emulsion may be created, where the substance is contained within the dispersed phase droplets, and the fiber-forming polymer is in the continuous phase. Surfactants may be used to stabilize the emulsion. The stable emulsion may then be electrospun. In additional examples, other forms of electrostatic spinning may be used, as will be apparent to a person of skill in the art after having the benefit of this disclosure, which may include without limitation, coaxial electrospinning, surface loading/post-spinning functionalization, or other techniques.

In one example, a substance 7912 that may include a medicinal product 1312 may be incorporated into nanofibers 7910 to create a solid dosage form 7900. In another example, a substance 8912 that may include a medicinal product 1312 and a pH adjustment composition 8914 may be incorporated into nanofibers 8910 to create a solid dosage form 8900. In this example, the gas may be additionally conditioned to control a pH balance of the gas delivered to the nasal passage, without limitation.

In another example, a substance 9912 that may include a medicinal product 1312 and one or more nutrient 9916 may be incorporated into nanofibers 9910 to create a solid dosage form 9900. As will be appreciated by those of skill in the art, excipients including nutrients 9916 may be included to stabilize the drug, aid in drug delivery, adjust tonicity, improve solubility, act as bulking agents, or control viscosity. Some substances derived from natural or nutrient sources may be used to enhance the absorption of drugs across the nasal mucosa. For example, a nutrient may open tight junctions between nasal epithelial cells to allow larger drug molecules to pass through more easily, form inclusion complexes with drug molecules to increase solubility and potentially enhancing their interaction with the nasal mucosa for absorption or otherwise increase the efficacy of delivery of the substance 9912 in the nasal cavity.

Additionally, in an example, a substance 10912 that may include a medicinal product 1312 and a molecular taggant 10918 may be incorporated into nanofibers 10910 to create a solid dosage form 10900. A molecular taggant 10918 may provide a chemical substance to identify, track, authenticate, or trace the material to which it is added. As will be appreciated by those of skill in the art, a molecular taggant 10918 may be designed to be detectable by spectroscopy, chromatography, fluorescence, or other analytical methods, without limitation.

The gas treatment component may additionally provide a physical-chemical identifier, for example an aroma, which may advantageously enhance a user's feeling of well-being and calmness, thus decreasing a user's stress. The aroma may, without limitation, apply a pleasurable or otherwise desirable scent to the air, increasing the mood and psychological well-being of a user operating an apparatus for conditioning a substance for delivery to a nasal cavity such as one enabled by this disclosure. In another embodiment, the physical-chemical identifier may provide essential oils, congestion relief solutions, and/or other substances that may be added to the gas prior to being sent to the nasal cavity of the user. In some embodiments, the aroma may be included by the solid dose form.

In one example, upon application of positive pressure by the gas source 1228, 5228, air may pass through the interior space of an apparatus for conditioning a substance for delivery to a nasal cavity such as one enabled by this disclosure sufficiently to come in contact with the aroma, thus transferring at least part of the aroma provided by the aromatic material to the gas. The aromatic gas may then pass through the nose plug component 1710, 2710 ,3710, 4710, 5710 and into the nasal cavity of a user engaging the olfactory receptors and providing a psychological and/or satisfactory response to the user.

The nose plug component will now be discussed in greater detail. FIGS. 1-6 highlight examples of the nose plug component, which may also be shown in other figures. The nose plug component 1710, 2710, 3710, 4710, 5710 may be operatively attached to the housing 1210, 2210, 4210, 5210 to be removably inserted into a nostril such to form an at least partial seal with a nasal cavity. The nose plug component 1710, 2710, 3710, 4710, 5710 may be constructed of a durable material to minimize potential damage when they are being handled, which material being substantially nontoxic to humans, for example silicon or graphene.

The nose plug component 1710, 2710, 3710, 4710, 5710 may be operatively installed to at least part of the housing 1210, 2210, 4210, 5210 provided by an apparatus for conditioning a substance for delivery to a nasal cavity enabled by this disclosure. The nose plug component 1710, 2710, 3710, 4710, 5710 may include a nose plug component 1710, 2710, 3710, 4710, 5710 having a nose plug base end 2722, 4722 and a nose plug nostril end 2724, 4724, 5724. The nose plug component 1710, 2710, 3710, 4710, 5710 may have a circumference and a diameter of a size such that, upon insertion into a nostril, the device may remain in place in the nostril anchored by friction. In one embodiment, the nose plug component 1710, 2710, 3710, 4710, 5710 may have a diameter of between approximately 8 millimeters and approximately 14 millimeters and a length between approximately 15 millimeters and approximately 25 millimeters, without limitation.

The nose plug component 1710, 2710, 3710, 4710, 5710 may include a nose plug aperture 2726, 3726, 4726 near the nose plug nostril end 2724, 4724, 5724. The nose plug component 1710, 2710, 3710, 4710, 5710 may additionally include a nose plug protruding lip near the nose plug base end 2722, 4722. Additional features may be provided by the nose plug that would be appreciated by those of skill in the art after having the benefit of this disclosure.

In one embodiment, provided without limitation, the nose plug component 1710, 2710, 3710, 4710, 5710 may be constructed using an at least partially flexible material, for example silicone, such that it may be inserted into the nostril of a user with minimal or negligible irritation. Additionally, by using a flexible material, the nose plug may provide an at least partial seal within the nostril such as to provide access to the nasal cavity of a user upon operation. The nose plug component 1710, 2710, 3710, 4710, 5710 may be configured such that the nose plug may be inserted at least partially into the nostril providing a pathway from the interior space provided by the housing 1210, 2210, 4210, 5210 through the nose plug component 1710, 2710, 3710, 4710, 5710 and into the nasal cavity. Accordingly, when in use, such nose plug components 1710, 2710, 3710, 4710, 5710 may extend into a nostril to a depth of between approximately 15 millimeters and approximately 25 millimeters, without limitation, as illustrated in FIG. 24.

The nose plug component 1710, 2710, 3710, 4710, 5710 may include a nose plug aperture 2726, 3726, 4726 that runs substantially throughout the interior of the nose plug. The nose plug aperture 2726, 3726, 4726 may extend substantially from the nose plug base end 2722, 4722 through the nose plug component 1710, 2710, 3710, 4710, 5710 to the nose plug nostril end 2724, 4724, 5724. Air and/or gas may be moved through the nose plug aperture 2726, 3726, 4726 passively through respiration, via pressure from gas source, and/or otherwise as would be appreciated by skilled artisans.

An embodiment where a substance 7912, 8912, 9912, 10912 is delivered via a nasal cavity from a substance source 1229 will now be discussed in greater detail. A substance delivery apparatus enabled by this disclosure may be used for nasal application of medicines, aroma therapy agents, gases, oxygen, and other substances, ensuring efficient and targeted delivery of a substance to the nasal cavity. A substance delivery apparatus may offer a versatile solution for nasal delivery of a wide range of substances, including pressurized oxygen, air, gases, medications, decongestants, and physical-chemical identifiers including aromatherapy solutions. The substance delivery apparatus may be configured to receive a substance from a substance source, such as an air delivery system of an aeronautical vehicle or space craft, air delivery source designed for military use, CPAP machine, medicine reservoir, aromatherapy diffuser, and/or other substance sources that would be apparent to a person of skill in the art after having the benefit of this disclosure. A substance delivery apparatus enabled by this disclosure may be designed to ensure targeted delivery, minimizing waste and maximizing the effectiveness of the substance being administered.

An apparatus enabled by this disclosure is particularly beneficial for individuals who require regular nasal administration of substances, such as those with chronic nasal congestion or allergies, or those that need pressurized oxygen delivery for medical, aeronautical, or astronautical application.

In some embodiments, the gas may be pressurized and substantially comprised of oxygen, which may originate from an on-board oxygen generation system of aeronautical and/or astronautical vehicles, a continuous positive airway pressure (CPAP) machine, and/or another source type that would be appreciated by a person of skill in the art after having the benefit of this disclosure. The substance may comprise a medicine comprising a medicinal product created by pharmaceutical formulation, which may be held by a reservoir optionally lined with nanofibers that may be constructed of a nonbiodegradable polymer, a solid nasal dose form, and/or other forms that would be appreciated by skilled artisans. In one example, at least part of the medicinal product is extracted from the solid dosage form to be combined with at least part of the gas in the delivery component to be delivered to the nasal cavity via the nose plug.

The substance delivered by the apparatus may include a physical-chemical identifier, such as aromatherapy agent, essential oils, or other fragrant compounds. These agents, when inhaled, can interact with the olfactory system, potentially influencing mood, emotions, and cognitive function. Such agents may be delivered for aromatherapy as a complementary treatment for psychological disorders. Aromatherapy has been explored as a potential therapeutic approach for various psychological conditions, including anxiety, depression, and stress-related disorders. For example, aromas can promote relaxation, reduce anxiety, and improve mood. By incorporating aromatherapy agents into a substance delivery apparatus enabled by this disclosure, an avenue for delivering therapeutic aromas may be provided directly to the nasal cavity, potentially enhancing their effectiveness and providing a non-invasive complementary treatment option for individuals with psychological disorders.

The substance delivered by an apparatus enabled by this disclosure may include nutrients, such as vitamins, minerals, or other nutritional compounds. These nutrients, when administered intranasally, can be absorbed through the nasal mucosa and enter the bloodstream, providing a direct pathway for nutrient delivery. This method bypasses the digestive system, potentially increasing bioavailability and offering a rapid alternative for nutrient supplementation. By delivering nutrients directly to the nasal cavity, an apparatus enabled by this disclosure advantageously offers a potential avenue for increasing absorption and bioavailability of administered nutrients, potentially leading to more efficient nutrient supplementation. This approach may be particularly beneficial for individuals with digestive disorders or those who have difficulty absorbing nutrients through the gastrointestinal tract.

In operation, an apparatus for conditioning a substance for delivery to a nasal cavity enabled by this disclosure may be used for purifying and/or reducing undesirable materials from air intended to enter a nasal cavity. Those of skill in the art will appreciate methods of use enabled by the above disclosure. Skilled artisans will appreciate additional methods within the scope and spirit of the disclosure for performing the operations provided by the examples herein after having the benefit of this disclosure. Such additional methods are intended to be included by this disclosure.

Referring now to flowchart 11000 of FIG. 11, an illustrative operation of conditioning a substance for delivery to a nasal cavity will now be discussed, without limitation. Starting at Block 11002, the operation may begin by mixing the active drug ingredients. (Block 11010). An excipient may then be mixed with the active drug ingredients to create a medicinal product. (Block 11012). The active drug ingredients and the excipient that are mixed may be stored in a solid dosage form, for example, as incorporated into nanofibers. (Block 11014). The medicinal product may then be mixed with gas to condition the substance. (Block 11016). The substance that is conditioned may then be delivered with the gas to the nasal cavity. (Block 11018). The operation may then terminate at Block 11020.

A substance delivery apparatus enabled by this disclosure may provide a versatile design with potential applications spanning various fields, including military, aerospace, and healthcare sectors. In military settings, the apparatus could be invaluable for personnel operating in hazardous environments with airborne contaminants. The filtration component, capable of removing fine particulates, could protect soldiers from inhaling dust, smoke, or even chemical warfare agents. The addition of a delivery component could allow for the delivery of prophylactic or therapeutic agents directly to the nasal passages, potentially mitigating the effects of inhaled toxins.

For astronautical applications, such as may be associated with NASA, an apparatus enabled by this disclosure could be adapted for use in spacecraft or during extravehicular activities. The filtration system could remove harmful dust particles or microorganisms present in extraterrestrial environments, while the ability to deliver supplemental oxygen or other gases could prove crucial in emergency situations. The compact and lightweight design would make it ideal for integration into spacesuits or other life support systems.

An apparatus enabled by this disclosure may also be compatible with continuous positive airway pressure (CPAP) machines, offering a potential upgrade to existing CPAP therapy. By incorporating the substance delivery, gas purification, and treatment components, an apparatus enabled by this disclosure could enhance the quality of air delivered to the user, potentially improving comfort and therapeutic outcomes. The addition of aromatherapy or delivery components could further personalize the CPAP experience, promoting relaxation and adherence to therapy.

While various aspects have been described in the above disclosure, the description of this disclosure is intended to illustrate and not limit the scope of the invention. The invention is defined by the scope of the appended claims and not the illustrations and examples provided in the above disclosure. Skilled artisans will appreciate additional aspects of the invention, which may be realized in alternative embodiments, after having the benefit of the above disclosure. Other aspects, advantages, embodiments, and modifications are within the scope of the following claims.

What is claimed is:

1. A substance delivery apparatus for nasal use comprising:
a housing to substantially enclose an interior space;
a nose plug component operatively attached to a nose end of the housing to be removably inserted into and form an at least partial seal with a nostril such that a substance comprising a medicinal product is directed to a nasal cavity through the nose plug component via the nostril; and
a delivery component located within the interior space to condition the substance and deliver the substance that is conditioned to the nasal cavity via the nose plug component.

2. The apparatus of claim 1, wherein conditioning the substance comprises mixing the substance with a gas, the apparatus further comprising:
a cap removably attached to a cap end of the housing to receive the gas delivered from a gas source via a delivery line, the cap end being distal to the nose end;
a gas treatment component located within the interior space to at least partially purify the gas for delivery to the nasal cavity comprising a filtration component to remove impurities from the gas; and
a cannula of the cap to receive the delivery line;
wherein the delivery line has a delivery line diameter configured to be selectively installed over the cannula such that the gas from the delivery line is received through a gas receiving aperture of the cannula.

3. The apparatus of claim 2, wherein the filtration component conditions the gas to at least partially filter nanoplastics from the gas received via the delivery line; and
wherein the nanoplastics being filtered by the filtration component have at least one dimension measuring less than 100 nanometers.

4. The apparatus of claim 2, wherein the gas is pressurized and substantially comprised of oxygen.

5. The apparatus of claim 4, wherein the oxygen originates from an on-board oxygen generation system of an aeronautical vehicle.

6. The apparatus of claim 2, wherein the gas is pressurized and originates from a continuous positive airway pressure (CPAP) machine.

7. The apparatus of claim 1:
wherein the substance further comprises a physical-chemical identifier;
wherein the medicinal product is stored via a solid dosage form; and
wherein at least part of the medicinal product is extracted from the solid dosage form to be mixed with a gas in the delivery component to be delivered to the nasal cavity via the nose plug.

8. The apparatus of claim 7, wherein the medicinal product is at least partially incorporated into the substance via a multi-step process of pharmaceutical formulation comprising the steps:
mixing active drug ingredients;
mixing an excipient with the active ingredients to create the medicinal product; and
mixing the medical product with the gas to condition the substance for delivery to the nasal cavity.

9. The apparatus of claim 8, wherein the pharmaceutical formulation further comprises the steps:
mixing a pH adjuster composition with the medicinal product to condition a pH level of the substance; and
creating the solid dosage form of the medicinal product from which the substance is delivered with the gas through the nose plug component that has been inserted into to the nostril.

10. The apparatus of claim 8, wherein the pharmaceutical formulation further comprises the step:
storing the active drug ingredients and the excipient that are mixed as the medicinal product in the solid dosage form.

11. The apparatus of claim 8, wherein the pharmaceutical formulation further comprises the step:
incorporating the substance in nanofibers via electrostatic spinning.

12. The apparatus of claim 8, wherein the pharmaceutical formulation further comprises the step:
storing the active drug ingredients and the excipient that are mixed with a physical-chemical identifier as the medicinal product in the solid dosage form; and
wherein the physical-chemical identifier initiates a psychological response in a patient after being received by the nasal cavity via the nose plug component.

13. The apparatus of claim 11, wherein the physical-chemical identifier comprises an aromatherapy agent.

14. The apparatus of claim 11, wherein the physical-chemical identifier comprises a molecular taggant.

15. The apparatus of claim 1:
wherein the substance comprises nutrients as absorption enhancers; and
wherein an efficacy of delivery of the substance via the nasal cavity is improved via the nutrients.

16. A substance delivery apparatus for nasal use comprising:
an interior space substantially enclosed by a housing;
a nose plug component operatively attached to a nose end of the housing to be removably inserted into and form an at least partial seal with a nostril such that a substance comprising a medicinal product is directed to a nasal cavity through the nose plug component via the nostril;
a delivery component located within the interior space to condition the substance and deliver the substance that is conditioned to the nasal cavity via the nose plug component;
a cap removably attached to a cap end of the housing to receive the gas delivered from a gas source via a delivery line, the cap end being distal to the nose end;
a gas treatment component located within the interior space to at least partially purify the gas for delivery to the nasal cavity comprising a filtration component to remove impurities from the gas; and
a cannula of the cap to receive the delivery line;
wherein the substance further comprises a physical-chemical identifier;
wherein the medicinal product is stored via a solid dosage form; and
wherein at least part of the medicinal product is extracted from the solid dosage form to be mixed with a gas in the delivery component to be delivered to the nasal cavity via the nose plug.

17. The apparatus of claim 16, wherein conditioning the substance comprises mixing the substance with a gas, the apparatus further comprising:
wherein the filtration component conditions the gas to at least partially filter nanoplastics from the gas received via the delivery line; and
wherein the nanoplastics being filtered by the filtration component have at least one dimension measuring less than 100 nanometers.

18. The apparatus of claim 16, wherein the medicinal product is at least partially incorporated into the substance via a multi-step process of pharmaceutical formulation comprising the steps:
mixing active drug ingredients;
mixing an excipient with the active ingredients to create the medicinal product;
storing the active drug ingredients and the excipient that are mixed as the medicinal product in the solid dosage form; and
mixing the medical product with the gas to condition the substance for delivery to the nasal cavity.

19. The apparatus of claim 18, wherein the pharmaceutical formulation further comprises the step:
incorporating the substance into nanofibers via electrostatic spinning.

20. The apparatus of claim 16:
wherein the substance comprises nutrients as absorption enhancers; and
wherein an efficacy of delivery of the substance via the nasal cavity is improved via the nutrients.
